(19) United States
(12) Patent Application Publication
(10) Pub. No.: US 2015/0275169 A1
(43) Pub. Date: Oct. 1, 2015

(54) PROCESS FOR THE LARGE SCALE PRODUCTION OF FRUIT CELLS

(71) Applicant: BIO HARVEST LTD., Rehovot (IL)

(72) Inventors: Yoheved Hagay, Rehovot (IL); Yoav Roth, Ashdod (IL); Malkit Azachi, Rehovot (IL); Rivka Yatuv, Shoham (IL)

(73) Assignee: Bio Harvest Ltd. a corporation

(21) Appl. No.: 14/439,667

(22) PCT Filed: Oct. 28, 2013
(86) PCT No.: PCT/IL2013/050877

§ 371 Date: Apr. 30, 2015

Related U.S. Application Data
(60) Provisional application No. 61/719,966, filed on Oct. 30, 2012.
(60) Provisional application No. 61/764,549, filed on Feb. 14, 2013.
(60) Provisional application No. 61/878,652, filed on Sep. 17, 2013.

(51) Int. Cl. C12N 5/04; A61K 31/05

(52) U.S. Cl. CPC C12N 5/04; A61K 31/05

(57) ABSTRACT

The invention relates to a large scale process for the in vitro production of a cell line callus culture of grape berry cells grown, to a product made according to this process and to a composition comprising a complex of pholyphenols including resveratrol, wherein the ratio of the resveratrol to the pholyphenols is higher than the ratio of resveratrol to pholyphenols in grapes grown naturally.

### FIELD OF THE INVENTION

This invention is directed to a process for the large scale production of fruit cells. One embodiment of the invention is directed to a process for the large scale in vitro production of fruit cells, which include primary and secondary metabolites.

### BACKGROUND OF THE INVENTION

Large scale processes are known in the art and are necessary for the industrial production of various materials. Since large scale processes cannot be performed by the same means as small scale processes, specific processes for the large scale production of materials must be designed, even if small scale processes exist.

Nutraceuticals are sometimes prepared using synthetic processes that provide the desired active ingredients, e.g., polyphenols, which are naturally found in fruit cells. However, the use of synthetic processes does not provide the natural ingredients along with the active ingredients, which sometimes contribute to the efficiency of the formulation.

Other types of nutraceuticals are prepared from the natural plants; however, all known large scale processes for preparing nutraceuticals from plants include the extraction of the prepared plant cells in order to obtain the desired active ingredient. However, when plants containing polyphenols, for example, are extracted, the final product may be bitter. Also, only certain parts of the plant may be successfully extracted since only they contain the desired amounts of the active ingredients.

Small scale processes for the preparation of fruit cells are known in the art; however, large scale processes are more difficult to design since they tend to amplify the production of the primary metabolites, while minimizing the productions of the secondary metabolites. Since active ingredients, such as polyphenols, are secondary metabolites their production in large-scale processes is complex.

Nutraceuticals derived from polyphenol-containing fruit extracts are known for their beneficial effects. The use of red wine as a source of these regulatory constituents is limited due to its high alcoholic content and sugar. In addition, it has been shown that the therapeutic effect of wine and wine grapes is dependant on species, location, year (annual climate), processing etc. and therefore reliance on red wine, grapes or grape seeds as a source of these regulatory compounds does not lead to a homogeneous or consistent supply of material. Furthermore, fruits are often contaminated by residual fungicides, pathogens, pesticides and pollutants.

Moreover, the potential benefit of gastrointestinal delivery of polyphenols from red wines and fruit extracts is limited by its bioavailability to target tissues and cells. Due to marked differences in their bioavailability while passing through the intestines, no correlation can be drawn between the abundance of a certain polyphenol in a given food and its concentration as an active compound in vivo. Absorbance of flavonoids in the small intestines, for example, ranges from 0-60% of the dose, and elimination half-lives range from 2-48 hours. Most polyphenols undergo extensive metabolism in the intestine, and are present in serum and urine predominantly as glucuronides, methyl or sulfate. Among the known polyphenols, the phytoalexin resveratrol (trans-3,5,4′-trihydroxystilbene) (RES), found in red grapes, red wine and other foods such as different kind of berries and peanuts has drawn most of the attention. It is believed to be responsible for the “French paradox”, a phenomenon associated with low incidence of cardiovascular diseases despite high-fat diet as a result of moderate red wine consumption. However, RES bioavailability is compromised by its physicochemical properties such as low water solubility and also its high hepatic uptake. Moreover, oral bioavailability of RES is extremely low due to rapid and extensive metabolism with the consequent formation of various metabolites.

Studies investigating RES activity and effects rely mainly on three sources of resveratrol, namely pure synthetic RES, natural plant-derived RES (e.g. Poligonum cupcidatum extracts) products, or to a lesser extent whole red grapes or their products (red wine, grape juice, grape extracts). Red Grape Cells (RGC; Fruitura Bioscience Ltd, Israel) is a natural patent-protected formulation of cultured cells originated from the fruits of Vitis Vinifera L. cultivar comprising the whole matrix of polyphenols including resveratrol and other ingredients naturally existing in red grape.

The aromatic groups in RES structure enable it to function as antioxidant and prevent important reactions in diseases processes such as LDL oxidation occurring in atherosclerosis. RES was also shown to modulate the inflammatory responses underlying chronic diseases such as cancer and diabetes. Animal studies have shown RES involvement in attenuation of pain as well as acute inflammation.

Thus, there is a need in the art for a large scale process for preparing fruit cells from natural ingredients, which includes the production of both the primary and the secondary metabolites of the fruit cells. There is need for natural (phyto) compositions that may be prepared in a large scale process in which the amount of the active ingredient is consistent and recurrent (e.g., clonal preparations), is highly bioavailable and easily administered for the treatment and prevention of various diseases and disorders.

### SUMMARY OF THE INVENTION

In an embodiment of the invention, there is provided a large scale process for the in vitro production of a cell line callus culture of grape berry cells grown comprising: growing grape cells in a flask; inoculating the grape cells from the flask into a first bioreactor; inoculating the grape cells from the first bioreactor into another bioreactor, wherein the another bioreactor is a last bioreactor or an intermediate bioreactor and wherein at least one of the first and the another bioreactor is disposable; and harvesting the grape cells from the last bioreactor; wherein the grape cells harvested from the last bioreactor are dried.

In some embodiments of the invention, the size of each bioreactor used in the process is larger than the one in which the grape cells were previously grown.

In some embodiments of the invention, if the another bioreactor is an intermediate bioreactor, an additional step of inoculating the grape cells to another intermediate bioreactor or to the last bioreactor in performed.

In some embodiments of the invention, the first or another bioreactor is a 4-10, 10-50, 50-200, 200-1000 or 1000-5000 liter bioreactor.

In some embodiments of the invention, the grape cells are grown in a Gamborg B5 medium.

In some embodiments of the invention, the Gamborg B5 medium is enriched with magnesium, phosphate or nitrate salts or a combination thereof.

In some embodiments of the invention, the Gamborg B5 medium is enriched with KNO₃, MgSO₄, MgNO₃ or NaH₂PO₄ or a combination thereof.

In some embodiments of the invention, the disposable bioreactor is made from one or more layers of polyethylene.

In some embodiments of the invention, the disposable bioreactor is made from an inner and outer layer of polyethylene and a middle nylon layer.

In some embodiments of the invention, the Gamborg B5 medium does not include plant hormones.

In some embodiments of the invention, the Gamborg B5 medium includes plant hormones.

In some embodiments of the invention, the Gamborg B5 medium is enriched with 2-4% sucrose.

In an embodiment of the invention, there is provided a composition in a form of a powder comprising a cell line callus culture of grape berry cells grown in vitro in a large scale up process, whereby the cell line callus culture of grape berry cells is derived from one or more of grape-berry cross section, grape-berry skin, grape-berry flesh, grape seed, grape embryo of seeded or seedless cultivars or grape seed coat; wherein the cell line callus culture of grape berry cells includes resveratrol in an amount of at least 1000 mg/kg powder.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 demonstrates measurements of paw edema (volume %) in rates treated with RGC preparation, indomethacin and water as a control before carrageenan injection. Statistical analysis was carried out using two-way ANOVA for repeated measures, followed by Bonferroni post hoc tests. Comparison of control group (1M) to positive control group (2M) showed statistically significant difference at 2 and 4 h (p<0.001). Comparison of control group to RGC-preparation (3M) group showed statistically significant difference at 2 and 4 hours (p<0.001).

FIG. 2 shows the distribution of group's hyperalgesic effect measured by Hot Plate during the study (the results are expressed as “Hot plate latency, % from baseline” as a function of time). Statistical analysis was carried out using two-way ANOVA for repeated measures, followed by Bonferroni post hoc tests. Comparison of control group (1M) to positive control group (2M) showed statistically significant difference at 2 and 4 h (p<0.05-0.01). Comparison of control groups RGC (3M) showed statistically significant difference at 4 h (p<0.01).

FIG. 3 shows the distribution of group's hyperalgesic effect measured in Hot Plate Test (the results are expressed as “Hot plate latency delta, baseline-actual time” as a function of time). Statistical analysis was carried out using two-way ANOVA for repeated measures, followed by Bonferroni post hoc tests. Comparison of control group (1M) to positive control group (2M) showed statistically significant difference at 2 and 4 h (p<0.05-0.01). Comparison of control groups and RGC-treated mice (3M) showed statistically significant difference at 4 h (p<0.01).

FIG. 4 presents growth curves of four different batches of Red Grape Cells grown in a large scale disposable bioreactor in enriched Gamborg B5 medium. The cells undergo exponential growth yielding a 500 gr/1 fresh biomass at day 20 up to 40.

FIG. 5 provides results for the resveratrol (RES) solubility in water, comparing the solubility of RGC-RES, synthetic-RES and plant-RES.

FIGS. 6A and 6B present the plasma contents of trans-RES after the administration of single dose of RGC RES, wherein FIG. 9A presents the total trans-RES and FIG. 9B presents free trans-RES. The presented values are means (n=15).

### DETAILED EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Embodiments of the invention are directed to a process for the large scale in vitro production of fruit cells. In Some embodiments of the invention, the process does not include the extraction of the fruit cells. Surprisingly, the produced fruit cells manufactured in accordance with the large scale process described herein were shown to include high amount of polyphenols particularly, the second metabolite resveratrol. The invention provides a unique composition of red grapes cells (RGC) which as an outcome of scale up process, includes of a whole matrix of polyphenols and other healthy ingredients naturally existing in red grape cells, with significantly higher concentration of grape resveratrol (40 to 800 fold or more higher) than the concentration is found in fresh grapes (see experiment 9 and table 9).

As used herein the term “polyphenols” refers to naturally occurring phyto organic compounds having more than one phenol group. Polyphenols may range from simple molecules, such as phenolic acid, to large, highly polymerized, compounds such as tannins. The phenolic rings of polyphenols are typically conjugated to various sugar molecules, organic acids and/or lipids. Differences in this conjugated chemical structure account for the chemical classification and variation in the modes of action and health properties of the various polyphenol compounds. Examples of polyphenols include, but are not limited to, anthocyanins, bioflavonoids (including the subclasses flavones, flavonols, isoflavones, flavanols, and flavanones), proanthocyanins, xanthones, phenolic acids, stilbenes and lignans. Resveratrol (3,4,5-trihydroxystilbene), which is one type of the polyphenols is a polyphenolic stilbene appears in its monomers forms; trans-resveratrol, cis-resveratrol, trans-glucoside and cis-glucoside.

According to an embodiment of the invention, the fruit is a grape. The grape may be a colored grape (e.g. red, black, purple, blue and all color variations between). Alternatively, the grape may be a non-colored grape (e.g. green or white or any color variation between).

The fruit of this aspect of the invention may be of a wild or cultivated variety. Examples of cultivated grapes include those grapes belonging to the vitis genus. Examples of vitis varieties include, but are not limited to, Vitis vinifera (V. vinifera), V. silvestris, V. muscadinia, V. rotundifolia, V. riparia, V. shuttleworthii, V. lubrisca, V. daviddi, V. amurensis, V. romanelli, V. aestivalis, V. Cynthiana, V. cineria, V. palmate, V. munsoniana, V. cordifolia, Hybrid A23-7-71, V. acerifolia, V. treleasei and V. betulifolia.

According to some embodiments, the fruit cells are derived from a colored or a non-colored grape. As described herein, according to some embodiments, the fruit cells are prepared from a fruit cell culture. According to some embodiments of the invention, the fruit cells are prepared from a culture of grape berry cells. According to some embodiments, the culture of grape berry cells is derived from one or more of grape-berry cross section, grape-berry skin, grape-berry flesh, grape seed, grape embryo of seeded or seedless cultivars or grape seed coat. According to another embodiment, the fruit cell culture may be derived from any part of a plant including, but not limited to endosperm, aleurone layer, embryo (or its parts as scutellum and cotyledons), pericarp, stem, leaves, tubers, trichomes and roots.

According to some embodiments of the invention, although the amount of materials, including polyphenols, may vary in fruit, the use of a culturing protocol for preparing the fruit cell cultures ensures the reproducibility of the preparation and its contents. Thus, various batches of fruit cells, prepared from the same culture have a typical HPLC fingerprint. According to some embodiments, the concentrations of the various materials in each batch may change, though, as mentioned above, if prepared from the same culture, the HPLC fingerprint is consistent for all batches.

In some embodiments of the invention, there is provided a composition in a form of a powder comprising a cell line callus culture of grape berry cells grown in vitro in a large scale up process, whereby the cell line callus culture of grape berry cells is derived from one or more of grape-berry cross section, grape-berry skin, grape-berry flesh, grape seed, grape embryo of seeded or seedless cultivars or grape seed coat; wherein the cell line callus culture of grape berry cells includes resveratrol in an amount of at least 1000 mg/kg powder. In some embodiments of the invention, at least 90% of the resveratrol in the grape cells manufactured in accordance with the embodiments of the large scale process described herein is trans-glucoside resveratrol.

According to some embodiments, the relative amounts of the various polyphenols in the prepared fruit cells, differ from the relative amounts thereof in the agricultural grape fruit. This can be clearly seen in Example 3, Table 9 in which the total resveratrol in dried grape cell culture produced in large scale in accordance with the embodiments of the invention was compared to the amount of the resveratrol in grapes. According to some embodiments, the amount of certain polyphenols is amplified in the prepared fruit cells, in comparison to their amount in the agricultural grape fruit. According to some embodiments, the amount of the resveratrol is amplified in the fruit cells. According to some embodiments, the amount of resveratrol in the fruit cells, which may be grape cells, is between 1000-50000 mg/kg, after the fruit cells, which may be grape cells are dried to a powder. According to some embodiments of the invention, the amount is more than 1000 mg/kg after the fruit cells are dried to a powder. According to some embodiments of the invention, the amount is more than 3000 mg/kg after the fruit cells are dried to a powder. According to some embodiments of the invention, the amount is more than 5000 mg/kg after the fruit cells are dried to a powder. According to some embodiments of the invention, the amount is more than 10000 mg/kg after the fruit cells are dried to a powder. According to some embodiments of the invention, the amount is more than 20000 mg/kg after the fruit cells are dried to a powder. According to some embodiments of the invention, the amount is more than 30000 mg/kg after the fruit cells are dried to a powder. According to some embodiments of the invention, the amount is more than 40000 mg/kg after the fruit cells are dried to a powder. According to some embodiments of the invention, the amount is more than 50000 mg/kg after the fruit cells are dried to a powder. According to some embodiments of the invention, the amount is more than 60000 mg/kg after the fruit cells are dried to a powder. According to some embodiments of the invention, the amount is more than 70000 mg/kg after the fruit cells are dried to a powder.

According to some embodiments, the relative amounts of various ingredients in the prepared fruit cells, differ from the relative amounts thereof in the agricultural grape fruit. According to some embodiments, the relative amount of sugar in the fruit cells is reduced in comparison to the relative amount of the sugar in the agricultural grape fruit.

According to some embodiments, the fruit cells prepared according to the large scale method of the invention contain less than 10% w/v sweetening sugar. According to some embodiments, the fruit cells contain less than 5% w/v sweetening sugar. According to some embodiments, the fruit cells contain less than 3% w/v sweetening sugar. According to some embodiments, the fruit cells contain less than 2% w/v sweetening sugar. According to some embodiments, the fruit cells contain less than 1% w/v sweetening sugar. According to some embodiments, the fruit cells contain about 1% w/v sweetening sugar. As used herein, the phrase “a sweetening sugar” refers to a sugar which provides a sweet taste e.g. sucrose, glucose and fructose.

According to some embodiments, the fruit cells are dried, thus concentrating the materials found therein, including the sugar. According to some embodiments, the materials are concentrated by a factor of 5. According to some embodiments, the materials are concentrated by a factor of 10. According to some embodiments, the materials are concentrated by a factor of 15. According to some embodiments, the materials are concentrated by a factor of 20. According to some embodiments, the materials are concentrated by a factor of 25. According to some embodiments, the materials are concentrated by a factor of 30.

According to one embodiment, the dried fruit cells contains up to 10% w/v sweetening sugar. According to some embodiments of the invention, the dried fruit cells contains up to 15% w/v sweetening sugar. According to one embodiment, the dried fruit cells contain between 10-15% w/v sweetening sugar. According to one embodiment, the dried fruit cells contain between 15-20% w/v sweetening sugar.

According to one embodiment, the dried fruit cells contain less than 20% w/v sweetening sugar. According to one embodiment, the dried fruit cells contain less than 30% w/v sweetening sugar.

According to some embodiments, the fruit cells prepared according to the large scale method of the invention are tasteless According to other embodiments, the fruit cells prepared according to the large scale method of the invention are tasteful.

In one embodiment of the invention, there is provided a large scale process for the in vitro production of a cell line callus culture of grape berry cells grown comprising:
growing grape cells in a flask;
inoculating the grape cells from the flask into a first bioreactor; and harvesting the produced grape cells.
   In some embodiments of the invention, there is provided a large scale process for the in vitro production of a cell line callus culture of grape berry cells grown comprising:
growing grape cells in a flask;
inoculating the grape cells from the flask into a first bioreactor; inoculating the grape cells from the first bioreactor into another bioreactor, wherein the another bioreactor is a last bioreactor or an intermediate bioreactor and there may be provided some more steps with one or more intermediate bioreactor and wherein at least one of the first and the another bioreactor is disposable; and
harvesting the grape cells from the last bioreactor;
wherein the grape cells harvested from the last bioreactor are dried.

By a “disposable bioreactor” it is meant a bioreactor with a disposable bag, which can be for a single use bag instead of a culture vessel. The disposable bag is typically made of three layers or more plastic foil. In some embodiments of the invention, one layer is made from polyethylene, polyethylene terephthalate or LDPE to provide mechanical stability. A second layer is made using nylon, PVA or PVC that acts as a gas barrier. Finally, a contact layer is made from PVA or PP or another layer of polythyelene, polyethylene terephthalate or LDPE. For medical applications the single-use materials that contact the product must be certified by the European Medicines Agency or similar authorities responsible for other regions.

According to some embodiments of the invention, the disposable bioreactor is made from one or more layers of polyethylene. In some embodiments of the invention, the disposable bioreactor is made from an inner and outer layer of polyethylene and a middle nylon layer.

In general there are two different approaches for constructing single-use bioreactors, differing in the means used to agitate the culture medium.

Some single-use bioreactors use stirrers like conventional bioreactors, but with stirrers that are integrated into the plastic bag. The closed bag and the stirrer are pre-sterilized. In use the bag is mounted in the bioreactor and the stirrer is connected to a driver mechanically or magnetically.

Other single-use bioreactors are agitated by a rocking motion. Other single-use bioreactors are airlift bioreactor in which the reaction medium is agitated and aerated by introduction of air. This type of bioreactor does not need any mechanical agitators inside the single-use bag.

According to some embodiments, the large scale process for preparing fruit cells is comprised of a number of subsequent steps. According to some embodiments of the invention, the amount of fruit cells prepared in each step is larger or not than that prepared in the previous step. Further, the fruit cells prepared in each step are inoculated or harvested to be used as a starter for the next step of the large scale process. In the last step of the large scale process, the fruit cells are typically grown until they reach the plateau in their growth profile.

The advantages of using the large scale process of the invention are clear and demonstrated in the Examples section. As can be seen in Example 2, experiment 2, the Red Grape Cells (RGC) biomass with enriched Gamborg B5 medium was much higher in comparison to the biomass obtained in the presence of non-enriched Gamborg B5 medium. Further, the use of Gamborg B5 medium containing different concentrations of magnesium, nitrates and phosphates (KNO₃, MgSO₄, MgNO₃, NaH₂PO₄) salts resulted in high resveratrol level in the produced cells, even in the large scale bioreactor.

Table 4 and Example 2, experiment 3 shows that the level of total polyphenols as well as of resveratrol in enriched medium, in the grape cells grown in the large scale disposable bioreactors was higher than the level obtained in the grape cells grown in Erlenmeyer flask, namely 910 mg/l and 203 mg/l, respectively. In contrast to observations made by others, this is the first time to demonstrate successful growth of fruit plant cells in large scale disposable bioreactor of 1000-5000 liter with high level of resveratrol and polyphenols production.

According to some embodiments, there is provided a composition comprising a complex of pholyphenols including resveratrol, wherein the amount of the resveratrol to polyphenols is higher than 1:20. In some embodiments, the ratio is higher than 1:10. In some embodiments, the ratio is higher than 1:5. In some embodiments, the ratio is higher than. In some embodiments, the ratio is higher than 1:3. In some embodiments, the ratio is higher than 1:2. According to some embodiments, the composition is derived from a natural source. According to some embodiments, the composition is derived from a natural source. According to some embodiments, the composition is derived from grape cells grown in large scale disposable bioreactors. According to some embodiments, the composition is derived from grape cells grown in large scale disposable bioreactors according to the process described herein.

Table 7 and Example 2, experiment 7 show the effect of two mediums, IM1 medium and Gamborg B5 enriched with magnesium, phosphate and nitrates salts on the amount of resveratrol produced by the cells. The effect was assessed in 20 L bioreactors made of a sterilized, disposable, transparent plastic container and further compared to data published in A. Decendit (1996) Biotechnology Letters, where cells were grown in IM1 medium in a 20 L glass container. The results demonstrate that Red Grape Cells grown in IM1 medium in a disposable bioreactor produced 93 mg/l of resveratrol (Table 7) which is approximately three fold higher than the level produced in stirred glass bioreactor using the same medium (Decendit). Moreover, significant high level of resveratrol, 387 mg/l, was produced when these cells were grown in enriched Gamborg B5 in disposable bioreactor. Thus, this experiment shows the advantages of using a disposable bioreactor or bioreactors and the Gamborg B5 medium containing different concentrations of magnesium, nitrates and phosphates (KNO₃, MgSO₄, MgNO₃, NaH₂PO₄) salts as well as the advantages of using the combination of a disposable bioreactor or bioreactors and an enriched Gamborg B5 medium.

According to some embodiments, the fruit cells are grown in bioreactors. According to some embodiments, the bioreactors are designed so as to allow adequate mixing and mass transfer, while minimizing the intensity of shear stress and hydrodynamic pressure. According to some embodiments of the invention, at least one of the bioreactors is a disposable bioreactor. This can be the first bioreactor or the intermediate bioreactor or the last bioreactor or any combination thereof. According to some embodiments of the invention, the disposable bioreactor is the last bioreactor after which the cells are harvested and dried so as to form a powder.

According to an exemplary embodiment of the invention, the first step includes the preparation of a fruit cell culture in a flask, such as an Erlenmeyer or a bioreactor. According to some embodiments, the first step involves the preparation of up to 1.0 L of a fruit cell culture. According to further embodiments, first step involves the preparation of up to 1.5 L of a fruit cell culture. According to further embodiments, first step involves the preparation of up to 2.0 L of a fruit cell culture.

According to some embodiments, the first step is conducted using a glass, metal or plastic flask. According to some embodiments, the flask is disposable. According to further embodiments, the flask may be reused any number of times. According to some embodiments, the flask is sterilized by any appropriate means between uses.

According to some embodiments, the first step includes the use of any appropriate medium for growing the fruit cells. According to some embodiments, the medium used for growing the fruit cells includes cell growth medium, salts, vitamins, sugars, hormones or any combination thereof. According to some embodiments, the cell growth medium is B5 Gamborg (Gamborg et al., Exp. Cell Res. 50:151, 1968), or any modification thereof. According to some embodiments, the Gamborg B5 comprises salts such as magnesium, phosphate, nitrate or any combination thereof. According to some embodiments of the invention, the Gamborg B5 medium includes KNO₃, MgSO₄, NaH₂PO₄, or any combination thereof.

According to some embodiments, the medium includes Gamborg B5 vitamins or any combination thereof. According to further embodiments, the medium includes sugars such as sucrose, Gamborg B5 or any combination thereof.

In an embodiment of the invention, the concentration of the KNO₃ added to the Gamborg B5 is between 25 mM to 45 mM.

In an embodiment of the invention, the concentration of the MgSO₄ added to the B5 Gamborg is between 1 mM to 15 mM.

In an embodiment of the invention, the concentration of the MgNO₃ added to the B5 Gamborg is between 5 mM to 35 mM.

In an embodiment of the invention, the concentration of the KNO₃ added to the Gamborg B5 is between 15 mM to 60 mM.

In an embodiment of the invention, the concentration of the MgSO₄ added to the B5 Gamborg is between 0.5 mM to 25 mM.

In an embodiment of the invention, the concentration of the MgNO₃ added to the B5 Gamborg is between 1 mM to 50 mM.

In an embodiment of the invention, the concentration of the KNO₃ added to the Gamborg B5 is between 30 mM to 40 mM.

In an embodiment of the invention, the concentration of the MgSO₄ added to the B5 Gamborg is between 5 mM to 10 mM.

In an embodiment of the invention, the concentration of the MgNO₃ added to the B5 Gamborg is between 20 mM to 30 mM.

In an embodiment of the invention, myo-inositol is added to the Gamborg B5

In an embodiment of the invention, H₃BO₃ added to the Gamborg B5

In an embodiment of the invention, MnSO₄ added to the Gamborg B5.

In an embodiment of the invention, NaH₂PO₄ is added to the Gamborg B5.

In an embodiment of the invention, Biotin is added to the Gamborg B5

In an embodiment of the invention, D-Pantothenate calcium is added to the Gamborg B5.

In an embodiment of the invention, about 0.5 mM myo-inositol is added to the Gamborg B5

In an embodiment of the invention, about 0.05 mM H₃BO₃ added to the Gamborg B5

In an embodiment of the invention, about 0.04 mM MnSO₄ added to the Gamborg B5.

In an embodiment of the invention, about 1 mM NaH2PO₄ is added to the Gamborg B5.

In an embodiment of the invention, about 0.004 mM Biotin is added to the Gamborg B5

In an embodiment of the invention, about 0.2 mM D-Pantothenate calcium is added to the Gamborg B5.

In an embodiment of the invention, about 0.1, 0.2, 0.3, 0.4, 0.5, 0.6, 0.7, 0.8, 0.9, 1.0, 2, 3, 4, 5, 6, 7, 8, 9, 10 mM myo-inositol is added to the Gamborg B5.

In an embodiment of the invention, about 0.01, 0.02, 0.03, 0.04, 0.05, 0.06, 0.07, 0.08, 0.09, 0.1 mM H₃BO₃ is added to the Gamborg B5.

In an embodiment of the invention, about 0.01, 0.02, 0.03, 0.04, 0.05, 0.06, 0.07, 0.08, 0.09, 0.1 mM MnSO₄ is added to the Gamborg B5.

In an embodiment of the invention, 0.1, 0.2, 0.3, 0.4, 0.5, 0.6, 0.7, 0.8, 0.9, 1.0, 2, 3, 4, 5, 6, 7, 8, 9, 10 mM NaH2PO₄ is added to the Gamborg B5.

In an embodiment of the invention, about 0.001, 0.002, 0.003, 0.004, 0.005, 0.006, 0.007, 0.008, 0.009, 0.01 mM Biotin is added to the Gamborg B5

In an embodiment of the invention, about 0.1, 0.2, 0.3, 0.4, 0.5, 0.6, 0.7, 0.8, 0.9, 1.0, 2, 3, 4 mM D-Pantothenate calcium is added to the Gamborg B5.

In an embodiment of the invention, the concentration of the sucrose added to the Gamborg B5 is between 2 to 4%. In another embodiment, the concentration is about 3%.

According to further embodiments, casein, casein hydrolysate or casein peptone may be included in the cell growth medium. According to further embodiments growth hormones may be included in the cell growth medium. According to further embodiments, the growth medium includes hormones. According to some embodiments the fruit cells are grown without the addition of hormones.

Examples of plant culture media that may be used according to some embodiments in one stage or more of the process, include, but are not limited to: Anderson (Anderson, In Vitro 14:334, 1978; Anderson, Act. Hort., 112:13, 1980), Chee and Pool (Sci. Hort. 32:85, 1987), CLC/Ipomoea (CP) (Chee et al., J. Am. Soc. Hort. Sci. 117:663, 1992), Chu (N.sub.6) (Chu et al., Scientia Sinic. 18:659, 1975; Chu, Proc. Symp. Plant Tiss. Cult., Peking 43, 1978), DCR (Gupta and Durzan, Plant Cell Rep. 4:177, 1985), DKW/Juglans (Driver and Kuniyuki, HortScience 19:507, 1984; McGranahan et al., in: Bonga and Durzan, eds., Cell and Tissue Culture in Forestry, Martinus Nijhoff, Dordrecht, 1987), De Greef and Jacobs (De Greef and Jacobs, Plant Sci. Lett. 17:55, 1979), Eriksson (ER) (Eriksson, Physiol. Plant. 18:976, 1965), Gresshoff and Doy (DBM2) (Gresshoff and Doy, Z Pflanzenphysiol. 73:132, 1974), Heller's (Heller, Ann. Sci. Nat. Bot. Biol. Veg. 11th Ser. 14:1, 1953), Hoagland's (Hoagland and Amon, Circular 347, Calif. Agr. Exp. Stat., Berkeley, 1950), Kao and Michayluk (Kao and Michayluk, Planta 126:105, 1975), Linsmaier and Skoog (Linsmaier and Skoog, Physiol. Plant. 18:100, 1965), Litvay's (LM) (Litvay et al., Plant Cell Rep. 4:325, 1985), Nitsch and Nitsch (Nitsch and Nitsch, Science 163:85, 1969), Quoirin and Lepoivre (Quoirin et al., C. R. Res. Sta. Cult. Fruit Mar., Gembloux 93, 1977), Schenk and Hildebrandt (Schenk and Hildebrandt, Can. J. Bot. 50:199, 1972), White's (White, The Cultivation of Animal and Plant Cells, Ronald Press, NY, 1963), etc.

According to some other exemplary embodiments, the fruit cells and the medium are continuously mixed during the first step. According to further embodiments, the fruit cells and the medium are mixed occasionally during the first step. According to some embodiments, the temperature during the first step is between 20° C. and 30° C. According to some embodiments, the temperature during the first step is between 22° C. and 28° C. According to some embodiments, the fruit cells are grown in the first step for more than 5 days. According to some embodiments, the fruit cells are grown in the first step for more than 7 days. According to some embodiments, the fruit cells are grown in the first step for more than 5 days and less than 2 weeks. According to some embodiments, the fruit cells are grown in the first step for more than 5 days and less than 12 days.

According to some exemplary embodiments, the bioreactor used in the process of invention includes an inlet through which the fruit cells from the first step, the medium and any additional materials are placed into the bioreactor. According to further embodiments, the bioreactor used in the process of the invention includes an outlet for removing any materials desired. According to some embodiments, the outlet includes a gas outlet, designed to relieve the bioreactor of excess gases. According to some embodiments, the gas outlet is operated manually. According to other embodiments, the gas outlet is operated automatically, wherein gases are let out of the flask once the atmosphere in the flask reaches a pre-defined pressure. According to some embodiments, the predefined pressure up to 8 PSI.

Once the first step of the fruit cell growth is concluded, according to some exemplary embodiments, the fruit cells are inoculated into a small scale bioreactor, which is termed here also the first bioreactor. For the second step of the large scale process. According to some embodiments, the small scale bioreactor is a 4 L reactor. According to further embodiments, the small scale bioreactor is a 3-5 L reactor. According to further embodiments, the small scale bioreactor is a 3-10 L reactor. According to further embodiments, the small scale bioreactor is a 4-8 L reactor.

The small scale bioreactor may be prepared from any appropriate material, such as glass, metal, plastic and/or any type of polymer. According to some embodiments, the small scale bioreactor is disposable. If the small scale bioreactor is not disposable, according to some embodiments, it is cleaned and sterilized between uses by any appropriate means.

As described above, the production of secondary metabolites, including polyphenols, such as resveratrol, is known to be significantly reduced when larger quantities of fruit cells are grown in bioreactors, in comparison to the amount of the same metabolites in small scale productions, using, e.g., glass flasks, such as Erlenmeyers. However, the large scale process detailed herein provides fruit cells in which the amount of the secondary metabolites is not reduced when grown in bioreactors. Further, the production of certain secondary metabolites may even be amplified.

Thus, according to embodiments of the invention, the relative amounts of the secondary metabolites in fruit cells grown in the small scale bioreactor are not significantly reduced in comparison to their relative amounts in the first step of the process. According to some embodiments, the components described above for use in the growth medium in the first step may be used also in the second step of the process. According to some embodiments, the growth medium used in the small scale bioreactor is the same as used in the first step of the large scale process. According to some embodiments, the relative amounts of the different components found in the growth medium in the second step, is the same as in the first step. According to other embodiments, the relative amounts of the different components found in the growth medium in the second step, differ from the relative amounts used in the first step. According to some embodiments, additional materials are added to the growth medium in the second step of the process.

According to some embodiments, the small scale bioreactor includes an inlet through which the fruit cells from the first step, air, the medium and any additional materials are placed into the bioreactor. According to further embodiments, the small scale bioreactor includes an outlet for removing any materials desired. According to some embodiments, the outlet includes a gas outlet, designed to relieve the bioreactor of excess gases. According to some embodiments, the gas outlet is operated manually. According to other embodiments, the gas outlet is operated automatically, wherein gases are let out of the bioreactor once the atmosphere in the bioreactor reaches a pre-defined pressure. According to some embodiments, the predefined pressure is 8 PSI.

According to some embodiments, the fruit cells and the medium are continuously mixed during the second step. According to further embodiments, the fruit cells and the medium are mixed occasionally during the second step. According to some embodiments, the temperature during the second step is between 20 to 30 Celsius degrees. According to some embodiments, the fruit cells are grown in the second step for more than a week and less than two weeks. In some embodiments of the invention, the fruit cells are grown between 9-16 days before being inoculated into the next bioreactor.

For the third step of the large scale process, the harvested fruit cells are placed into a large scale bioreactor. According to some embodiments, the large scale bioreactor is a 30-50 L reactor. According to further embodiments, the large scale bioreactor is a 40-60 L reactor. According to further embodiments, the large scale bioreactor is a 30-70 L reactor. According to further embodiments, the large scale bioreactor is a 20-100 L reactor.

The large scale bioreactor may be prepared from any appropriate material, such as glass, metal, plastic and/or any type of polymer. According to some embodiments, the large bioreactor is disposable. If the large scale bioreactor is not disposable, according to some embodiments, it is cleaned and sterilized between uses by any appropriate means.

Similarly to the small scale bioreactor, according to embodiments of the invention, the relative amounts of the secondary metabolites in fruit cells grown in the large scale bioreactor are not significantly reduced in comparison to their relative amounts in any of the previous steps of the process. According to some embodiments, the components described above for use in the growth medium in any of the previous steps may be used also in the third step of the process. According to some embodiments, the growth medium used in the large scale bioreactor is the same as used in any of the previous steps of the large scale process. According to some embodiments, the relative amounts of the different components found in the growth medium in the third step, is the same as in any of the previous steps of the process. According to other embodiments, the relative amounts of the different components found in the growth medium in the third step, differs from the relative amounts used in any of the previous steps of the process. According to some embodiments, additional materials are added to the growth medium in the third step of the process.

According to some embodiments, the large scale bioreactor includes an inlet through which the fruit cells from the second step, the medium, air and any additional materials are placed into the bioreactor. According to further embodiments, the large scale bioreactor includes an outlet for removing any materials desired. According to some embodiments, the outlet includes a gas outlet, designed to relieve the bioreactor of excess gases. According to some embodiments, the gas outlet is operated manually. According to other embodiments, the gas outlet is operated automatically, wherein gases are let out of the bioreactor once the atmosphere in the bioreactor reaches a pre-defined pressure. According to some embodiments, the predefined pressure is up to 8 PSI.

According to some exemplary embodiments, the fruit cells and the medium are continuously mixed during the third step. According to further embodiments, the fruit cells and the medium are mixed occasionally during the third step. According to some embodiments, the temperature during the third step is between 20 and 30. According to some embodiments, the fruit cells are grown in the third step for about two to three weeks. According to some embodiments, the fruit cells are grown in the third step for about three to five weeks. According to some embodiments, the fruit cells are grown in the third step for about 12 to 30 days.

Once the third step of the fruit cell growth is concluded, the fruit cells are inculcated from the medium scale bioreactor typically by any appropriate means. For the fourth exemplary step of the large scale process, the harvested fruit cells are placed into a larger scale bioreactor. According to some embodiments, the larger scale bioreactor is a 1000 L reactor. According to further embodiments, the larger scale bioreactor is a 200-500 L reactor. According to further embodiments, the large scale bioreactor is a 500-1000 L reactor. According to further embodiments, the large scale bioreactor is a 1000-1500 L reactor. According to further embodiments, the large scale bioreactor is a 500-1100 L reactor.

The larger scale bioreactor may be prepared from any appropriate material, such as glass, metal, plastic and/or any type of polymer. According to some embodiments, the large scale bioreactor is disposable. If the large scale bioreactor is not disposable, according to some embodiments, it is cleaned and sterilized between uses by any appropriate means.

Similarly to the small scale bioreactors, according to embodiments of the invention, the relative amounts of the secondary metabolites in fruit cells grown in the larger scale bioreactor are not significantly reduced in comparison to their relative amounts in the previous steps of the process. According to some embodiments, the components described above for use in the growth medium in any of the previous steps may be used also in the fourth step of the process. According to some embodiments, the growth medium used in the larger scale bioreactor is the same as used in any of the previous steps. According to some embodiments, the relative amounts of the different components found in the growth medium in the fourth step, is the same as in any of the previous steps. According to other embodiments, the relative amounts of the different components found in the growth medium in the fourth step, differs from the relative amounts used in any of the previous steps. According to some embodiments, additional materials are added to the growth medium in the fourth step of the process.

According to some embodiments, the larger scale bioreactor includes an inlet through which the fruit cells from the third or second step, the medium and any additional materials are placed into the bioreactor. According to further embodiments, the larger scale bioreactor includes an outlet for removing any materials desired. According to some embodiments, the outlet includes a gas outlet, designed to relieve the bioreactor of excess gases. According to some embodiments, the gas outlet is operated manually. According to other embodiments, the gas outlet is operated automatically, wherein gases are let out of the bioreactor once the atmosphere in the bioreactor reaches a pre-defined pressure.

According to some embodiments, the fruit cells and the medium are continuously mixed during the fourth step. According to further embodiments, the fruit cells and the medium are mixed occasionally during the fourth step. According to some embodiments, the temperature during the fourth step is between 20 to 30 According to some embodiments, the fruit cells are grown in the third or fourth step until they reached a cell biomass of 10% to 70%.

According to some embodiments, the large scale process is terminated after the fruit cells are grown in the larger scale bioreactor. According to such embodiments, the fruit cells are grown in the larger scale bioreactor until they reach a cell biomass of 10% to 70%. Once the cell biomass of 10% to 70%. is reached, the fruit cells are harvested from the large scale bioreactor by any appropriate means and are further processed. According to some embodiments, the fruit cells are further processed by drying, lyophilization, Freeze-Drying and Spray Drying. According to some embodiments, the processing of the fruit cells does not include the extraction of active ingredients therefrom.

According to some embodiments, the large scale process may include one step of inoculating the cells from a flask into a bioreactor which can be in any size and harvesting the cells. According to other embodiments, the fruit cells may be inculcated in a series of bioreactors wherein each of the bioreactors is typically larger than the previous bioreactor used. Any number of additional steps is performed according to the large scale process. The additional steps include possible intermediate steps in which the cells are harvested or inoculated and placed in a larger bioreactor and grown there until being harvested or inoculated and transferred to a larger bioreactor. According to further embodiments, the process includes additional steps for growing the fruit cells harvested from the large scale bioreactor.

In an embodiment of the invention, there is provided a pharmaceutical or nutraceutical composition or a food additive comprising the grape cells manufactured in the large scale process of the invention. The pharmaceutical or nutraceutical composition or a food additive may be administered to the subject by oral administration.

As used herein, the phrase “pharmaceutical composition” refers to a preparation of fruit cell culture, which may be a grape cells culture as further described hereinabove with or without other chemical components such as physiologically suitable carriers and excipients.

In an embodiment of the invention, there is provided a method of treating an inflammatory disorder by administering to a subject in need a pharmaceutical or nutraceutical composition or a food additive comprising the grape cells cell culture manufactured large scale process in accordance with the embodiments of the invention.

As used herein the term “treating” refers to the prevention of some or all of the symptoms associated with an inflammatory disease, a condition or disorder. The term “treating” also refers to alleviating the symptoms or underlying cause of an inflammatory disease, prolongation of life expectancy of patients having a disease, as well as complete recovery from a disease.

As used herein the phrase “inflammatory disorder” includes but is not limited to chronic inflammatory diseases and disorders and acute inflammatory diseases and disorders. This is exemplified in Example 4 which shows that the paw edema and behavioral hyperalgesia associated with carrageenan-induced hind paw inflammation in rats were positively attenuated by the oral administration of red grape cells (RGC) manufactured according to the large scale process described herein being indicative of the anti-inflammatory effect RGC that are prepared in a large scale process.

Various aspects of the invention are described in greater detail in the following Examples, which represent embodiments of this invention, and are by no means to be interpreted as limiting the scope of this invention.

### EXAMPLES

### Example 1

### Manufacturing-Industrial Level Scaling Up

### 1. Material and Methods

The production process encompasses propagation of grape cells in a progressively process having five stages. Starting from propagation of grape cells in an Erlenmeyer shake flasks for further propagation in a small and large scale disposable bioreactor. The critical key factor is maintaining a high level of the secondary metabolite, resveratrol in the cells during the propagation in different bioreactors scale. At the end of the last large scale stage of propagation, at the required biomass, the cells are harvested and dried to produce a fine pink-purple powder yielding a biomass of dried cells (RGC) which are used for different medicinal applications.

Grape Cell Line Formation

Calli from Grape Cross Sections:

Young grape bunches, 4 to 8 cm long, were harvested from field grown grape plants 20-50 days post anthesis and were thoroughly rinsed in running tap water. Green immature berries of the seedless grape Vitis vinifera cv. “AVNIR 825” (a cross between Agraman and Gamay red) were sterilized for 10 minutes in a solution containing 1.3% w/v sodium hypochlorite and (0.1% v/v) Tween 20, as a wetting agent. Explants were dissected, using a scalpel, into 2 to 3 mm long traversal sections under half-strength MS (Murashige and Skoog, 1962, Physiol Plant 15:473-497) liquid basal medium supplemented with filter-sterilized 1.7 mM ascorbic acid and 0.8 mM citric acid, 100 mg/l DTT (dithiothreitol) and 50 mg/l acetyl cysteine. The following antioxidants were added to the cutting medium: PVP (0.5 and 1 g/l), L-cysteine (150 mg/)l, gallic acid (1.5 mg/l), DTT (70 mg/l), biopterin (15 mg/l), ascorbic acid (150 mg/l) and citric acid (150 mg/l) in order to inhibit cell necrogenesis and to enable the recovery of green, health berry disks.

Berry disks were placed in 100×15 mm culture plates containing 25 ml of autoclaved Murashige and Skoog, MS medium, solidified with 0.25% Gelrite. The pH was adjusted to pH 5.9 prior to autoclaving at 102 kpa for 15 minutes. Thirty plates each containing 25 berry disks, were sealed with Parafilm and incubated in the dark at 26° C. for three days. Cultures were incubated at 25° C. under a 16-h photoperiod of 15-30 μmolm⁻² s⁻¹ irradiance provided by cool-white fluorescent tubes. MS salt and vitamins medium was also supplemented with 250 mg/l casein hydrolisate, 2% sucrose and 100 mg/l inositol. For callus induction it was also supplemented with 0.2 mg/l Kinetin and 0.1 mg/l NAA (α-naphthalenacetic acid) media designated as RD1.

3-4 weeks following culture initiation, a mixture of green and red callus was visible along the berry disks. The callus was composed of friable, elongated cells, some of which exhibited a dark pigmentation of anthocyanins. Callus sector enriched in anthocyanins were selected and individually subcultured for propagation. Green callus sectors were cultured separately.

Calli from Grape Skin Cells:

Young grape bunches, 4 to 8 cm long, were harvested 20-50 days post anthesis from field grown grape plants and were thoroughly rinsed in running tap water. Green immature berries of the seedless grape Vitis vinifera cv. “AVNIR 825” (a cross between Agraman and Gamay red) were sterilized for 10 minutes in a solution containing 1.3% w/v sodium hypochlorite and (0.1% v/v) Tween 20, as a wetting agent. Berry skins were isolated by producing a cut of 3-8 mm in the berry skin and peeling off only the skins using a sterile forceps. Skin isolation was performed under half-strength MS (Murashige and Skoog, 1962) liquid basal medium supplemented with filter-sterilized 1.7 mM ascorbic acid and 0.8 mM citric acid, 100 mg/l DTT (dithiothreitol) and 50 mg/l acetyl cysteine.

Berry skins were placed in RD-1 culture media. Following about 10-14 days, cell clumps started to develop on the cut surface of the skin pills. Cell, enriched in anthocyanins, were selected and further subcultured into fresh media for further propagation.

Calli from Grape Seed Coats:

Young grape bunches, 4 to 8 cm long, were harvested 20-50 days post anthesis from field grown grape plants and were thoroughly rinsed in running tap water. Green immature berries of the seedless grape Vitis vinifera cv. “AVNIR 825” were sterilized for 10 minutes in a solution containing 1.3% w/v sodium hypochlorite and (0.1% v/v) Tween 20, as a wetting agent. Berries were cut open to reveal the young green developing seeds. Immature seed coats were dissected and placed on culture medium. Isolation was performed under half-strength MS (Murashige and Skoog, 1962) liquid basal medium supplemented with filter-sterilized 1.7 mM ascorbic acid and 0.8 mM citric acid, 100 mg/l DTT (dithiothreitol) and 50 mg/l acetyl cysteine.

The seed coat sections were placed in RD-1 culture media. After about 12-20 days, seed coats turned brown and a callus started to appear on top of the seed coat explants. Cell, enriched in red-brown pigmentation, were selected and further subcultured into fresh media for further propagation.

Establishment of Liquid Cultures:

Liquid cultures were established by addition of 10 g of callus into 50 ml of the different media (RD1-RD6—see below). All cell lines that were successfully established on solid media developed a homogenous cell suspension in the same media combinations but lacking a gelling agent. The addition of 70 mg/l DTT or 150 mg/l of either ascorbic acid or citric acid improved growth and inhibited cell necrogenesis of berry derived suspension culture. All explant types were successfully utilized for the establishment of liquid cultures. Cultures were routinely subcultured every 7-10 days to fresh growing media.

Additional Vitis Species that were Introduced in Order to Establish Berry Derived Callus Cell Lines:

The following Vitis species were cultured using the above mentioned protocol:

Vitis silvestris, V. muscadinia, V. rotundifolia, V. riparia, V. shuttleworthii, V. lubrisca, V. daviddi, V. amurensis, V. romanelli, V. aestivalis, V. Cynthiana, V. cineria, V. palmate, V. munsoniana, V. cordifolia, Hybrid A23-7-71, V. acerifolia, V. treleasei, V. betulifolia.

The efficiency of callus production of Vitis vinifera grape cross sections, grape skins and grape seeds is exemplified in Table 1 hereinbelow.

TABLE A

| | Number of plate | % of plated | Type of callus |
|---|---|---|---|
| Explant type | cultured | producing calli | produced |
| Berry disks | 537 | 64 | Light red to |
| | | | purple |
| Skin | 498 | 51 | Dark red to |
| | | | purple |
| Seed coat | 428 | 49 | Red brownish |

The efficiency of production of the different callus ‘types’ from some of the Vitis species utilized in this study is summarized in Table 2 hereinbelow.

TABLE B

| | | Average efficiency of | | |
|---|---|---|---|---|
| | Explant | callus production | Callus | Optimal |
| Vitis species | type | (%) | type | Media |
| Muscadinia | B SC, S | 43 | Dark red | RD1 |
| (rutondifolia) | | | | |
| shuttleworthii | B SC, S | 29 | Red | RD3 |
| aestivalis | B SC, S | 36 | Dark red | RD2 |
| Hybrid A23-7-71 | B SC, S | 19 | Red- | RD6 |
| | | | brownish | |
| amurensis | B SC, S | 51 | Red | RD1 |

(B—Berry disk, SC—Seed coat, S—Skin)

Stage 1: Erlenmeyer, Shake Flasks

Red Grape Cells are grown in suspension under continuous fluorescent light (1000 1×) at 25±5° C., in 1 liter Erlenmeyer flasks on an orbital shaker. The growing medium contains Gamborg B5 medium and vitamins and is supplemented with 250 mg/l casein hydrolizate, 2-4% sucrose, 100 mg/l Myo-inositol, 0.2 mg/l Kinetin and 0.1 mg/l NAA (1-naphthaleneacetic acid), 25-45 mM KNO₃, 1-15 mM MgSO₄ or 5-35 mM MgNO₃ and 1 mM NaH₂PO₄ (pH 5.8). The cells are sub-cultured every 6-11 days.

Stage 2: Small Scale Bioreactor

Small scale bioreactor culturing is performed by inoculating a 7 to 16 old day cell suspensions grown in the Erlenmeyer of stage 1 into a 4-8 liter disposable bioreactor at 25±5° C. The cells are grown in the suspension under continuous fluorescent light (1000 1×) in a growing medium containing enriched Gamborg B5 salt and vitamins medium supplemented with 250 mg/l casein hydrolizate, 2-4% sucrose, 100 mg/l Myo-inositol, 25-45 mM KNO₃, 1-15 mM MgSO₄ or 5-35 mM MgNO₃ and 1 mM NaH₂PO₄, 0.2 mg/l Kinetin and 0.1 mg/l NAA (1-naphthaleneacetic acid) (pH 5.4-5.8). The cells are sub-cultured every 9-16 days.

Stage 3: Large Scale Bioreactor

The cell suspension grown in a small scale bioreactor are inoculated into a 30-50 liter disposable bioreactor. The cells are grown in a suspension under continuous fluorescent light (1000 1×) at 25±5° C. The growing medium containing enriched Gamborg B5 salt and vitamins medium supplemented with 250 mg/l casein hydrolisate, 2-4% sucrose, 100 mg/l Myo-inositol, 25-45 mM KNO₃, 1-15 mM MgSO₄ or 5-35 mM MgNO₃ and 1 mM NaH₂PO₄, 0.2 mg/l Kinetin and 0.1 mg/l NAA (1-naphthaleneacetic acid) (pH 5.4-5.8). The cells are sub-cultured every 12-30 days.

Stage 4: Larger Scale Bioreactor

The cell suspension grown in a small or large scale bioreactor are inoculated into a 300-1000 liter disposable bioreactor. The cells are grown in a suspension under continuous fluorescent light (1000 1×) at 25±5° C. The growing medium contains enriched Gamborg B5 salt and vitamins medium supplemented with 250 mg/l casein hydrolisate, 2-4% sucrose, 100 mg/l Myo-inositol, 25-45 mM KNO₃, 1-15 mM MgSO₄ or 5-35 mM MgNO₃ and 1 mM NaH₂PO₄, 0.2 mg/l Kinetin and 0.1 mg/l NAA (1-naphthaleneacetic acid) (pH 5.4-5.8).

Stage 5: Harvesting

The cells are harvested once they reach a cell biomass of 10% to 70% (w\v). The harvested cells are dried to produce a fine pink-purple powder, with a typical composition, taste and odor.

### Example 2

### The Effect of Medium Composition and Bioreactor Design on the Level of Resveratrol in Red Grape Cells Grown in Large Scale Bioreactor

### 2.1 Medium Composition

### Experiment 1: The Effect of Medium Composition on the Amount of Resveratrol in Red Grape Cells Grown in Erlenmeyer Shake Flask.

Red Grape Cells were grown in Erlenmeyer shake flask as described on Example 1, stage 1, in different medium compositions: MS, WP, WP+ casein hydrolysate (casein peptone) Gamborg B5. The results presented in Table 1, demonstrate that cells grown in the presence of Gamborg B5 medium produce approximately 4 to 15 fold higher resveratrol levels than cells grown in the presence of MS, WP and WP+ Casein hydrolysate (casein peptone) medium.

TABLE 1

| | | | Total |
|---|---|---|---|
| | Fresh weight | Resveratrol | Polyphenols |
| Medium* | g/L | mg/L | mg/L |
| MS** | 125 | 13.1 | 143.4 |
| WP*** | 132 | 24.6 | 208.9 |
| WP+ Csaein | 152 | 7.7 | 126.2 |
| hydrolizate | | | |
| Gamborg B5 | 166 | 141 | 735 |

*The data are the mean of at least two experiments
**MS—Murashige and Skoog medium (Toshio Murashige and Folke K. Skoog in 1968)
***WP—Woody Plant Medium (WP) (Lloyd and McCown, 1981)

### Experiment 2: The Effect of Medium Composition on the Growth and Resveratrol Level in Red Grape Cells Grown in a Large Scale Disposable Bioreactor.

Red Grape Cells were grown in large disposable bioreactor as described on Example 1 stage 3, in different medium compositions.

Red Grape Cells were grown in two types of medium: Gamborg B5 medium and enriched Gamborg B5. As revealed in Table 2 below, supplementation of Gamborg B5 medium with high level of magnesium, phosphate and nitrates or sulfate salts (KNO₃, MgSO₄, MgNO₃, NaH₂PO₄) resulted in a higher Red Grape Cells biomass in comparison to the biomass obtained in the presence of non-enriched Gamborg B5 medium.

TABLE 2
Growth of Red Grape Cells in scale up bioreactors in Gamborg B5 and
enriched Gamborg B5 medium

| | Fresh weight |
|---|---|
| Medium composition | gram/L |
| Enriched Gamborg B5 | 240 |
| (KNO₃, MgSO₄, MgNO₃) | |
| Gamborg B5 | 110 |

* The data are the mean of three experiments

In addition, the effect of medium composition on resveratrol and total polyphenols levels in Red Grape Cells grown in a large scale disposable bioreactor was examined. The cells were grown in WP medium and in Gamborg B5 medium containing different concentrations of magnesium, nitrates and phosphates (KNO₃, MgSO₄, MgNO₃, KNO₃ and NaH₂PO₄, accordingly) salts. Table 3 demonstrates that these salts are required for production of high levels of polyphenols and of resveratrol, in particular when added to the enriched Gamborg B5 medium. As opposed in WP medium, the level of total polyphenols and resveratrol is very low.

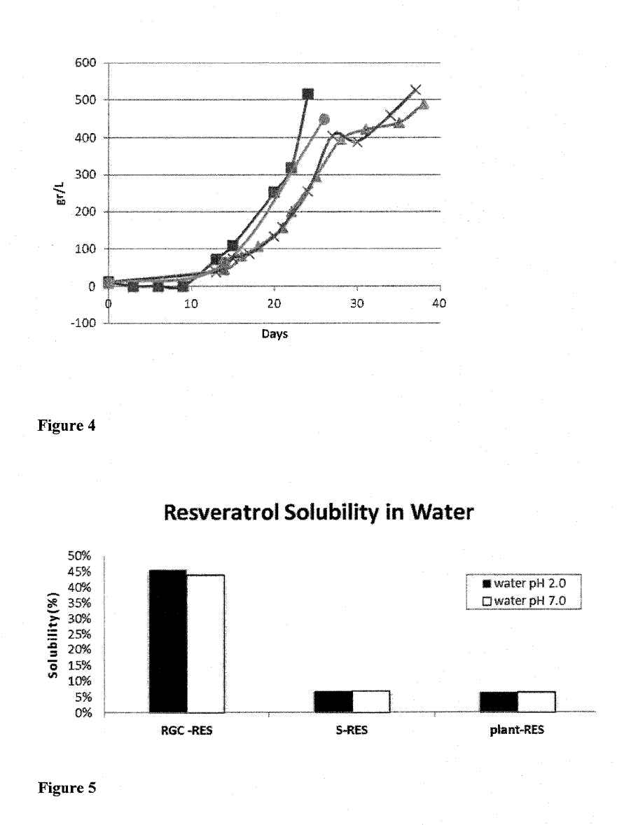

FIG. 4 presents growth curves of Red Grape Cells grown in a large scale disposable bioreactor in enriched Gamborg B5 medium. The cells undergo exponential growth yielding a 500 gr/l fresh biomass at day 20 up to 40. These cells continue to grow and can reach a higher biomass.

TABLE 3
Levels of Resveratrol, and total polyphenols in RGC grown in different mediums
with different levels of salts, in large scale bioreactors (50 L)

| | | | | | | Total |
|---|---|---|---|---|---|---|
| | | | | | Resveratrol | Polyphenols |
| | | Treatment | | | eq. Trans- | eq. |
| | MgSO₄ | MgNO₃ | NaH₂PO₄ | KN0₃ | resveratrol | Epicatechin |
| Medium*** | mM * | mM * | mM * | mM * | μg/mg | μg/mg |
| **B5 | | (5-5.7) | 2 (1) | 50 (25) | 18.0-25.3 | 41.4-54.1 |
| **B5 | | (5.5-15) | 2 (1) | 50-70 | 12.4-22.5 | 31.6-63.5 |
| | | | | (25-45) | | |
| **B5 | | (20-25) | 2 (1) | 70 (45) | 12.7-16.8 | 35.2-44.1 |
| **B5 | 9.1 (8) | (5) | 2 (1) | 50 (25) | 13.5-18.4 | 29.8-67.4 |
| **B5 | 9.1 (8) | | 2 (1) | 50 (25) | 15.0-20.3 | 42.6-48.1 |
| **B5 | 13.1 (12) | | 2 (1) | 50 (25) | 14.0-31.3 | 51.4-117.9 |
| **B5 | 16.1 (15) | | 2 (1) | 50 (25) | 13.2-18.5 | 34.0-53.0 |
| **B5 | 13.1 (12) | (25-35) | 2 (1) | 50 (25) | 6.2-16.8 | 40.5-44.1 |
| ****WP | | | | | 1.0 | 15.2 |

Note-
The values in part present as a range
* Numbers in bracket describe additional concentrations of minerals added to Gamborg B5
**The data are the mean of at least of three experiments.
***The data are the mean of at least ten experiments.
****McCown's Woody Plant medium (Lloyd and McCown, Proc. Int. Plant Prop. Soc. 30: 421, 1981

Experiment 3: Consistency of Resveratrol and Total Polyphenols Levels in RGC Grown in Different Growth Stages from Erlenmeyer Shake Flask to Large Scale Bioreactor

Red Grape Cells were grown in different scale stages from Erlenmeyer shake flask to a large scale disposable bioreactor in the presence of enriched Gamborg B5 as described on Example 1, stages 1 to 4.

The results in table 4 show that Red Grape Cells grow well in Erlenmeyer and synthesized high amount of resveratrol and total polyphenols. When the cells were grown in either 50 liter, 300 to 1000 liters scale in disposable bioreactors, a higher growth rate achieved in comparison to the growth in an Erlenmeyer flask as revealed by fresh and dry weight of the cells, see the data in Table 4. In all sizes of the scale up bioreactors, the fresh weight was above 230 g/l (Table 4) as compared to 166 g/l in Erlenmeyer. Moreover, the level of total polyphenols as well as of resveratrol in the enriched medium, in large scale disposable bioreactors was higher than the level obtained in the Erlenmeyer flask 901 mg/l and 200 mg/l, respectively (Table 4). In contrast to observations made by others, this is the first time to demonstrate successful growth of fruit plant cells in large scale disposable bioreactor with high level of resveratrol and polyphenols production.

TABLE 4
Resveratrol and total polyphenols level in RGC grown in shake flask
and different scale stages*

| | Fresh | Dry | | Total |
|---|---|---|---|---|
| | weight | weight | Resveratrol | Polyphenols |
| Experimental set-up | gram/L | gram/L | mg/L | mg/L |
| Erlenmeyer flask | 166 | 8.2 | 141 | 735 |
| Small scale | 102 | 15 | 179 | 1287 |
| disposable bioreactor | | | | |
| Large scale up 50 | 313 | 13 | 205 | 1087 |
| liters disposable | | | | |
| bioreactor | | | | |
| Larger scale | 242 | 15 | 200 | 901 |
| disposable bioreactor | | | | |
| 300-1000 liters | | | | |

*The data are the mean of at least of three experiments.

### Experiment 4: The Effect of Casein Hydrolyzate Addition on Resveratrol and Total Polyphenols Level in Red Grape Cells Grown in Large Scale Disposable Bioreactor

Red Grape Cells were grown in a large scale disposable bioreactor in the presence of enriched Gamborg B5 as described in Example, 1 stage 3.

As can be seen in Table 5, the levels of resveratrol and total polyphenols in Red Grape Cells were similar when grown in disposable large scale bioreactor in enriched medium with or without 250 mg/l casein hydrolisate.

TABLE 5

| | | Total |
|---|---|---|
| | Total resveratrol | Polyphenols |
| Medium | gr/kg dry powder | gr/kg dry powder |
| Without Casein hydrolizate* | 13.6 ± 2.2 | 45.4 ± 6.0 |
| With casein hydrolizate | 13.1 ± 1.6 | 63.2 ± 10.2 |

*The data are the mean of three experiments

### Experiment 5: The Effect of Plant Hormones Addition on Resveratrol and Total Polyphenols Levels on Red Grape Cells Grown in Large Scale Disposable Bioreactor.

Red Grape Cells were grown in a large scale disposable bioreactor in enriched Gamborg B5 as described on Example 1, stage 3. As can be seen in Table 6, the levels of resveratrol and total polyphenols production in Red Grape Cells were similar when grown in disposable large scale bioreactor with or without 0.5 mg/l NAA and 0.2 mg/l kinetin (Table 6).

TABLE 6

| | Total resveratrol | Total Polyphenols |
|---|---|---|
| Medium | mg/kg | mg/kg |
| Average ± SD | 12.0 ± 0.6 | 60.5 ± 3.0 |
| With hormones* | | |
| Average ± SD | 13.1 ± 1.6 | 63.2 ± 10.19 |
| Without hormones* | | |

*The data are the mean of three experiments

### Experiment 6: The Effect of Sucrose Concentration on Red Grape Cells Grown in Large Scale Disposable Bioreactor.

Red Grape Cells were grown in large disposable bioreactor (50 liter) as described on Example 1 stage 3, in enriched Gamborg B5 medium with different sucrose concentrations.

Red Grape Cells were grown in enriched Gamborg B5 mediums which contain 2, 3, 4 and 6% sucrose. As revealed in Table 6A below, optimum cell growth and biomass is achieved when cells are grown with 2 to 4% sucrose (143 to 260 gram/L). Higher sucrose concentration such as 6% sucrose inhibits cell growth by 10 fold (24 gram/L).

TABLE 6A

| | Fresh weight |
|---|---|
| Medium composition | gram/L |
| Enriched Gamborg B5 2% sucrose | 221 |
| Enriched Gamborg B5 3% sucrose | 260 |
| Enriched Gamborg B5 4% sucrose | 143 |
| Enriched Gamborg B5 6% sucrose | 24 |

### Experiment 7: The Effect of the Bioreactor Configuration, Design and Structure on Resveratrol and Total Polyphenols Levels in Red Grape Cells Grown in Large Scale Disposable Bioreactor

The effect of two mediums, IM1 medium and Gamborg B5 enriched with magnesium, phosphate and nitrates salts on the amount of resveratrol produced by the cells was assessed in 20 L bioreactors made from a sterilized, disposable, transparent plastic container and further compared to data published in A. Decendit (1996) Biotechnology Letters, where cells were grown in IM1 medium in a 20 L glass container.

The results demonstrated that Red Grape Cells grown in IM1 medium in a disposable bioreactor produced 93 mg/l of resveratrol (Table 7) which is approximately 3 fold higher resveratrol than the level produced in stirred glass bioreactor using the same medium (Decendit). Moreover, significant high level of resveratrol, 387 mg/l, was produced when these cells were grown in enriched Gamborg B5 in disposable bioreactor (Table 7).

TABLE 7
The effect of bioreactor type and medium composition on Resveratrol
levels produced by Red Grape cells

| | | Resveratrol |
|---|---|---|
| Medium | Bioreactor Type | mg/L |
| Enriched Gamborg B5 | Disposable | 387 |
| Medium** | | |
| IM1 Medium** | Disposable | 93 |
| IM1 Medium* | Stirred glass | 30 |

*IM1 Medium-Ref. article A. Decendit (1996) Biotechnology Letters
**The data are the mean of at least of three experiments

Further, the combination of specific medium composition that contains enriched Gamborg B5 and design disposable bioreactor induced the cells to produce 10 to 12 fold more resveratrol than the level that was produced in IM1 medium and stirred glass bioreactor and 4 fold more than in cell grown in IM1 medium in disposable bioreactor (table 7).

These results show that both the bioreactor type and the medium composition are required for maintaining high level of resveratrol in RGC produced in a bioreactor of 20 L or more.

### Example 3

### Composition

Both red and purple grapes contain powerful polyphenols, antioxidants and resveratrol, which helps to prevent both the narrowing and hardening of the arteries. Increasing research has shown that resveratrol found in red and purple grapes and ultimately in red wine—influences important metabolic pathways in the body and may benefit our health. They do, however, have a very high sugar content and should therefore be eaten in moderation.

The composition of Red Grape Cells grown in large scale disposable bioreactor is unique.

The chemical composition of Red Grape Cells is comparable to grapes grown using standard agricultural practices except from the level of the sugar and the resveratrol.

### Experiment 8: Lower Level of Total Sugars, Glucose and Fructose in Red Grape Cells Grown in Large Scale Disposable Bioreactor Compare to Red Grape Grown in the Vineyard.

The amount of total sugars, glucose and fructose in Red Grape Cells that were grown in a large scale disposable bioreactor as described on Example 1, stages 3 and 4 in enriched Gamborg B5 is 25 to 50 fold lower compare to the level of these sugars from deferent types of red grapes grown by agriculture means (table 8).

TABLE 8A & 8B
Sugars Comparison of sugars level between Red Grape Cells grown in large
scale disposable bioreactor and Agricultural Red Grapes (Fresh Weight)
(gr/100 gr)
A.

| | Agricultural Red Grapes | | | | |
|---|---|---|---|---|---|
| | Sample | Sample | Sample | Sample | |
| Sugars (%) | 1 | 2 | 3 | 4 | Range |
| Glucose | 7.47 | 10.71 | 11.32 | 8.69 | 7.4-11.3 |
| Fructose | 7.57 | 14.67 | 11.39 | 7.74 | 7.5-14.6 |
| Total sugars | 15 | 25.4 | 22.71 | 16.4 | 15-25.4 |

B.

| | RGC Batches | | | | | | | |
|---|---|---|---|---|---|---|---|---|
| Sugars (%) | 1 | 2 | 3 | 6 | 16 | 17 | 18 | Range |
| Glucose | 0.39 | 0.54 | 0.54 | 0.55 | 0.33 | 0.36 | 0.23 | 0.2-0.55 |
| Fructose | 0.311 | 0.42 | 0.46 | 0.36 | 0.43 | 0.73 | 0.25 | 0.31-0.73 |
| Total sugars | 0.7 | 0.96 | 1.0 | 0.91 | 0.76 | 1.1 | 0.48 | 0.48-1.1 |

sample 1—Agricultural Table Red Grape (Israel)—Red grape used for eating
Sample 2—Wine Red Grape 1 (Israel) Cabernet—Red grape used for making wine
Sample 3—Wine Red Grape 2 (Israel) Cabernet—Red grape used for making wine
Sample 4—Wine Red Grape 1 (South Africa) Cabernet—Red grape used for making wine

### Experiment 9: Level of Total Polyphenols and Resveratrol Composition of Red Grape Grown in Large Scale Disposable Bioreactor.

The levels of total polyphenols in Red Grape Cells are similar to the amounts in red grapes grown in the field with the exception of resveratrol, which is present is 40 to 800 fold higher (Tables 9 and 10). The level of resveratrol in five batches of Red Grape Cells grown in large scale disposable bioreactor as described on Example 1, stages 3 and 4, is in the range of between 726 to 916 mg/kg fresh weight compared to 1-12 mg/kg in agricultural red grapes (Table 9A, 9B). The level of resveratrol in the dry powder of Red Grape Cells after the drying process ranges from 6000 to 31000 mg/kg powder (Table 10).

Method: The levels of polyphenols and of Resveratrol in Red Grape Cells were analyzed using HPLC coupled with UV/VIS detection at 280, 520 and 306 nm.

TABLE 9A & 9B
Comparison of the Phenolic Content Composition
in Red Grape Cells and Agricultural Red Grape
9A. Phenolic Content Composition in Agricultural
Red Grapes (mg/kg fresh weight)

| | Agricultural Red Grapes |
|---|---|
| | Published Literature |
| | (mg/kg fresh weight) |
| Total | 131-361 ⁽¹⁾ |
| Polyphenols | 730-3480 ⁽²⁾ |
| | 1500-3900 ⁽⁴⁾ |
| Tannins | 1730-3480 ⁽²⁾ |
| (PACS) | 1600-4000 ⁽³⁾ |
| Resveratrol | 1-12 ⁽¹⁾ |

9B. Phenolic Content Composition in
Red Grape Cells (mg/kg fresh weight)

| | RGC Batches | | | | | |
|---|---|---|---|---|---|---|
| | 11 | 12 | 13 | 14 | 15 | Range |
| Total | 2333 | 2467 | 3133 | 2533 | 2600 | 2333-2600 |
| Polyphenols | | | | | | |
| Tannins | 2400 | 1867 | 3133 | 1840 | 2933 | 1840-3133 |
| (PACS) | | | | | | |
| Resveratrol | 813 | 916 | 906 | 866 | 726 | 726-916 |

⁽¹⁾ Cantos E, Epsin J C and Tomas-Barberan F A. Varietal Differences among the Polyphenol Profiles of Seven Table Grape Cultivars Studied by LC-DAD-MS-MS. J. Agric. Food Chem. 202, 50: 5691-5696.
⁽²⁾ Katalinic V, Mozina S S, Skroza D, Generalic I, Abramovic H, Milos M, Ljubenkov I, Piskernik S, Pwzo I, Terpinc P, Boban M, (2010). Polyphenolic profile, antioxidant properties and antimicrobial activity of grape skin extract of 14 Vitis Vinifera varieties grown in Dalmatia (Croatia). Food chemistry 119: 715-723.
⁽³⁾ Dell' Agli M, Busciala A, Bosisio E, (2004). Vascular effects of wine polyphenols. Cardiovascular research 63: 593-602.
⁽⁴⁾ Mattivi F, Zulian C, Nicolini G and Valenti L (2002). Wine, Biodiversity, Technology, and Antioxidants. Ann N.Y. Acad. Sci. 957: 37-56.

TABLE 10
Resveratrol and Total Polyphenolic compounds
content in Red Grape Cells (gr/kg dry powder)

| Polyphenol | Red Grape Cells batches | | | | | | | | | | |
|---|---|---|---|---|---|---|---|---|---|---|---|
| compounds | 1 | 2 | 3 | 4 | 5 | 6 | 17 | 18 | 7 | 8 | Range |
| Total | 83 | 30 | 43 | 60 | 101 | 68 | 45.7 | 46.0 | 29.3 | 43 | 29-101 |
| polyphenols | | | | | | | | | | | |
| Resveratrol | 31.3 | 20.4 | 13.6 | 24 | 18.1 | 14 | 17.6 | 16.8 | 14 | 6.3 | 6-31 |

### Example 4

### Effect of Cultured Grape Cells in In Vivo Carrageenan-Induced Paw Oedema Rat Model

The aim of the present study was to evaluate in vivo anti-inflammatory activity of RGC (Red Grape Cells) made according to the large scale process of the invention in an acute inflammation model in rats so as to verify the efficiency of the cells produced according to the invention. One of the experimental models most widely used to study acute inflammation in rodents is that based on the intraplantar administration of carrageenan.

The study assessed the efficacy of the RGC by two methods:
1. Paw volume measurements
2. Inflammatory nociception in freely moving rats

### Materials and Methods:

Red Grape Cells (RGC) prepared according to example 1.
RGC was administered orally at dose of 400 mg/kg body weight as a suspension in sterile drinking water, 2 h before carrageenan injection. The dose level was 40 mg/ml. Each rat was dosed with 1 ml of suspension per 100 g body weight.
RGC Composition: The amounts of polyphenols and resveratrol injected to each rat were 14 and 4.8 mg per body weight, respectively (Carrageenan induced rat paw edema: Rats were divided into three groups of eight rats in each group. The rats in all groups were injected with 1% Carrageenan (0.1 mg/paw) or sterile saline (0.9% NaCl) into the sub plantar tissue of left hind paw of each rat.
   The Following Tests were Performed:

### Paw Volumes Measurements

paw volumes were measured hourly just before carrageenan injection at time point 0 and 1, 2, 4 hours after injection using caliper.
The paw dimensions were measured in two axes and paw volume was calculated. Paw edema volume in this model was an indication of the inflammation severity.
For each time point, the change in paw volume was calculated either by subtracting the baseline paw volume or as % of baseline paw volume.

### Hot Plate Method for Measuring Inflammatory Nociception in Freely Moving Rats

The hot plate method was used to determine inflammatory nociception in freely moving rates. After inducing edema and treatment with vehicle, indomethacin or RGC preparation, the rats were placed on a hot plate maintained at a temperature of 55±0.5° C. The latency to flick or lick the hind paw or jump from the hot plate in comparison to its baseline was considered as the reaction time. The reaction time was noted at 1, 2, and 4 hours after injection. In absence of response, a 60 seconds cut off is used to prevent tissue damage.

### Group Allocation:

Vehicle control (1M) group: The control rats received sterile drinking water (vehicle), 2 h before carrageenan injection.
Positive control group (“2M”): the rats in the positive control group received 2 mg/kg body weight of indomethacin 2 h before carrageenan injection.
Test group (“3M”): the rats were administered orally with RGC at dose of 400 mg/kg body weight (1 ml/40 mg RGC per 100 gr body weight) as a suspension in sterile drinking water, 2 h before carrageenan injection.

### Results

### Paw Swelling:

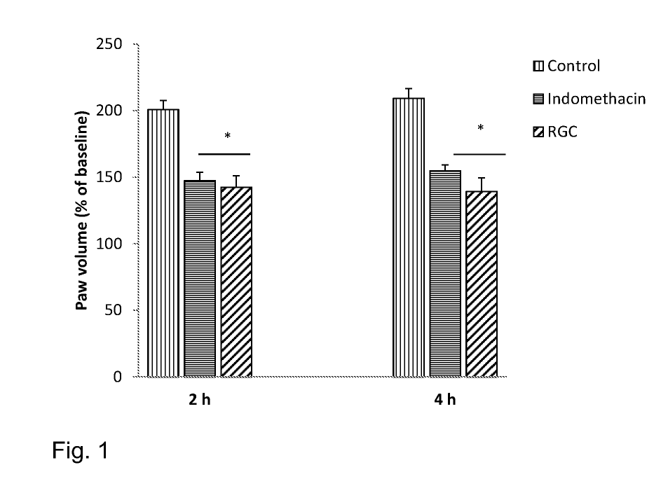

FIG. 1 shows the results of paw edema (volume %) in rates treated with RGC preparation, indomethacin and water as a control. Statistical analysis was carried out using two-way ANOVA for repeated measures, followed by Bonferroni post hoc tests. Comparison of control group (1M) to positive control group (2M) showed statistically significant difference at 2 and 4 h (p<0.001). Comparison of control group to RGC-preparation (3M) group showed statistically significant difference at 2 and 4 hours (p<0.001).

As shown in FIG. 1, paw swelling was reduced when the rats were treated with RGC preparation, at least to the level of the positive control, after 1 or 2 hours. Furthermore, after 4 hours the rats treated with RGC preparation demonstrated even a larger reduction in paw swelling then the control.

Inflammatory Nociception

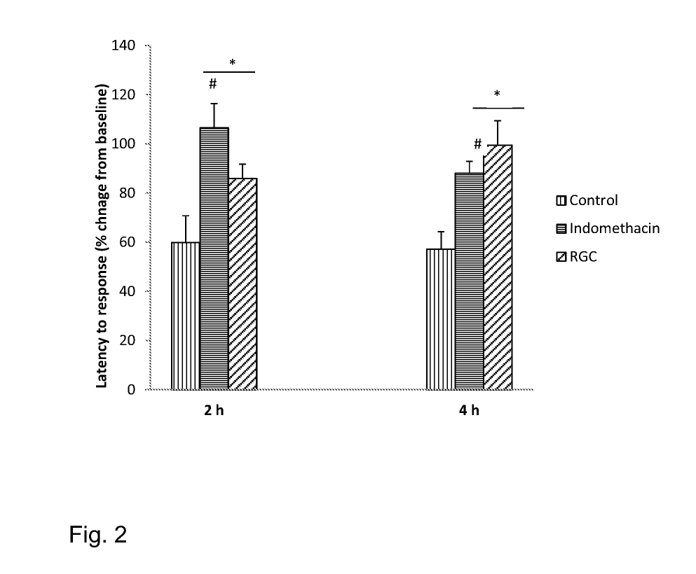

FIG. 2 shows the group's hyperalgesic effect following treatment with RGC preparation, indomethacin and water as a control. Statistical analysis was carried out using two-way ANOVA for repeated measures, followed by Bonferroni post hoc tests. Comparison of control group 1M to positive control group 2M showed statistically significant difference at 2 and 4 h (p<0.05-0.01). Comparison of control groups CGC (3M) showed statistically significant difference at 4 h (p<0.01).

As can be seen in FIG. 2, vehicle treated control group (1M)(received sterile drinking water) showed a significant increase in the latency delta (time) to the thermal stimulus, 2 and 4 hours after carrageenan injection. This was evidence by the decrease in reactivity to the thermal plantar stimulation of the rats in this group.

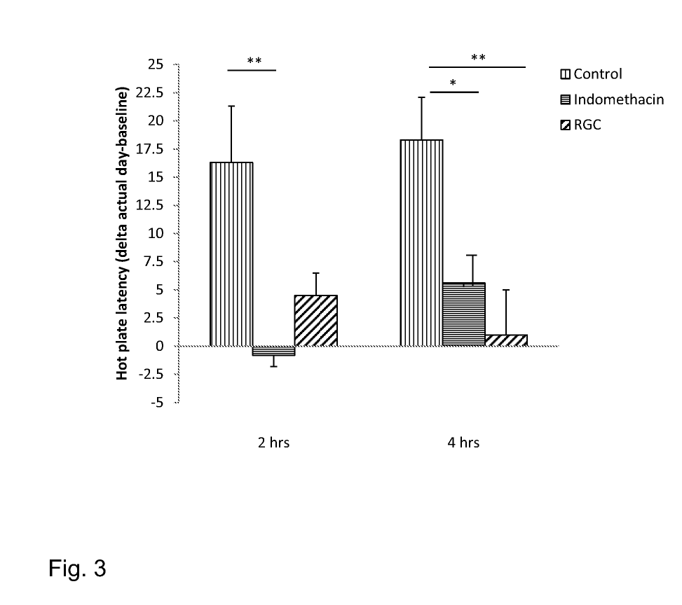

As can be seen in FIG. 3 Positive control group (indomethacin-treated rats, 2M) showed a significant decrease in the latency delta of response to the thermal stimulus, 2 and 4 hours after carrageenan injection compared to the vehicle control.

The RGC treated rats (3M) showed a decrease in the latency delta response to the thermal stimulus which, four hours after carrageenan injection, was statistically significant compared to vehicle treated controls.

In conclusion, the results demonstrated in this example show that the paw edema and behavioral hyperalgesia associated with carrageenan-induced hind paw inflammation in rats were positively attenuated by the oral administration of RGC made according to the process described in example 1 being indicative of the anti-inflammatory effect of the RGC preparation is restored even in RGC that are prepared in a large scale process.

### Example 5

### Chemical Properties of RGC-RES Compared to RES from Other Sources and Human Bioavailability Properties of RGC-RES

### Materials and Methods

Red Grape Cells (RGC) was prepared according to example 1. The resveratrol (RES) content of the RGC was determined by HPLC at 306 nm against a synthetic RES calibration curve.

### LC/MS Analysis of RGC-RES

RGC powder was dissolved in 80% methanol. Liquid chromatography mass spectrometry (LC-MS) analysis of the sample was performed using an Accela LC system coupled with the Linear Trap Quadrupole (LTQ) Orbitrap Discovery hybrid FT mass spectrometer (Thermo Fisher Scientific Inc.) equipped with an electrospray ionization source. The mass spectrometer was operated in the negative ionization mode and the mass spectra were acquired in the m/z 150-2000.

### Solubility Assay

RGC, synthetic resveratrol (S-RES; Sigma-Aldrich) and resveratrol extract from the plant Polygonum Capsidatum (plant-RES) were dissolved in 80% methanol to achieve 100% dissolution. Then, all RES sources were dissolved in water at pH 2 and pH 7 and the percentage of dissolution in water was compared to the dissolution in methanol. The RES in all samples was monitored at 306 nm, based on its characteristic absorption profile, and its concentration was determined by a calibration curve of resveratrol analytical standard.

### Results

### LC/MS

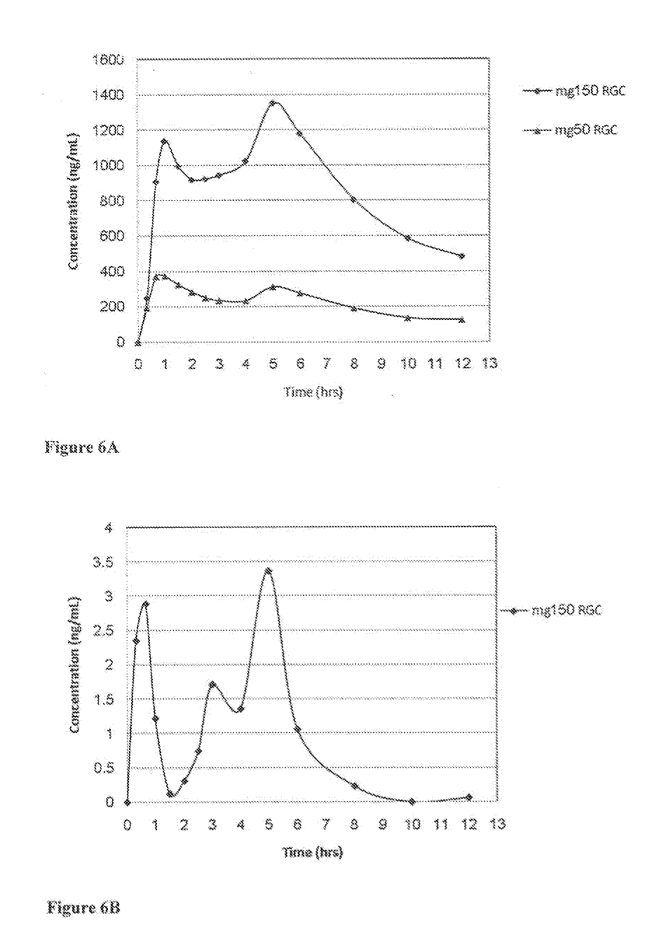

The mass spectra in negative ion mode and representative LC/MS chromatogram for the RGC powder are shown in FIGS. 6A and 6B. LC-MS analysis detected four derivatives of resveratrol (m/z −227.0701-227.0737) in RGC, all of which show UV absorbance at 306 nm. Three of these derivatives were hexose glycosides of trans-RES isomers, detected at retention times of 4.6, 5.3 and 6.1 min. The fourth derivative was of trans-RES, detected at a retention time of 6.9 min (Table 12). The identity of the four derivatives was confirmed, as shown by ESI mass spectrum (FIG. 7).

TABLE 11
Identification of resveratrol derivatives in RGC

| | Calculated | | |
|---|---|---|---|
| | Atomic | | |
| [M − H]⁻ | Composition | RT, min | Comment |
| 389.1250 | C₂₀H₂₁O₈ | 4.6 | Glycoside of trans-resveratrol |
| 389.1250 | C₂₀H₂₁O₈ | 5.3 | Glycoside of trans-resveratrol |
| 389.1250 | C₂₀H₂₁O₈ | 6.1 | Glycoside of resveratrol. |
| | | | Chromatographic separation was not |
| | | | complete but it also can be a |
| | | | derivative of cis-resveratrol |
| | | | (according to UV) |
| 227.0719 | C₁₄H₁₁O₃ | 6.9 | trans-Resveratrol |

### Solubility

The solubility of RGC-RES was compared to that of two trans-RES products: Synthetic-RES and plant Polygonum Capsidatum-RES. All three tested products were completely dissolved in 80% methanol solution. However, when the three RES products were dissolved in acidified water (pH=2), mimicking the stomach pH conditions as well as at pH 7.0, dissolution of RGC-RES was more than 6 fold higher, 44% for RGC-RES vs. 7% for the two other RES sources (FIG. 5).

### Human Bioavailability Study

The study was a single dose randomized, crossover comparative pharmacokinetic study. Fifteen adult healthy fasting male subjects received the investigational product RGC (oral doses equivalent to 50 mg or 150 mg of trans-RES separated by at least 7 day washout periods. A Standard meal was served 4 hours post dosing. The study was performed in compliance with all rules and regulations of the Israel Ministry of Health (MOH) and according to the ICH GCP guidance. The protocol was approved by the Soroka University Medical Center IRB and included administering a single dose of 50 or 150 mg to each patient, followed by a 7 day washout, a second single dose, which is different than the first dose, so that a patient who initially received 50 mg will receive 150 for the second dose and vice versa. Fifteen healthy non-smoking male volunteers were recruited into the study. Volunteer eligibility criteria included ages of 18 to 55 years; BMI ≧19 and ≦30; Subjects were asked to refrain from RES-containing food, nutritional supplements or drinks and from all drugs including over the counter medications from 7 days before the first dosing, and throughout the entire study period.

### Sample Collection and Management.

Venous blood samples were collected into K₂EDTA containing tubes before (t0) and at 0.33, 0.67, 1, 1.5, 2, 2.5, 3, 4, 5, 6, 8, 10 and 12 hours post dosing. Blood samples were kept in an ice bath and immediately handled under yellow light.

### Analysis of Resveratrol Content in Plasma

Sample preparation and LC-MS analysis for free and total RES in plasma samples was performed by PRACS Institute (Toronto, Canada). Plasma samples were liquid-liquid extracted and used for LC-MS/MS system for analysis. The lower limit of quantification (LLOQ) was 0.5 and 20 ng/mL for free and total RES, respectively. For the analysis of total RES, enzymatic hydrolysis and protein precipitation extraction was performed prior to LC-MS/MS analysis.

### Pharmacokinetic Analysis

The following pharmacokinetic variables were calculated for free RES and for total RES (free and conjugated) using a noncompartmental pharmacokinetic approach: maximal plasma concentration (Cmax) and time of maximal plasma concentration (Tmax), average concentration over the total collection period, the area under the plasma concentration versus time curve (AUC) from time (0) to the last quantifiable concentration (Clast, above LOQ) by the trapezoidal method.

### Results

Demographics and Safety:

Fifteen healthy male volunteers participated in the study. Subjects age range was 28 to 55 years (mean 42.1 years) and BMI range was 21.4 to 30 (mean 25.8). All subjects were tested negative to drugs and alcohol with no clinically significant abnormalities concerning laboratory parameters and vital sign measurements at screening and admission. No adverse events were observed or reported throughout the study.

Plasma Pharmacokinetics of Free and Total Resveratrol:

Mean trans-RGC-RES plasma concentration versus time curves for total RES and free RES is displayed in FIG. 9. As can be seen, RGC profile at both concentrations demonstrates two clear concentration peaks, the first after 1 hour and a second (higher) peak after 5 hours.

Mean pharmacokinetic parameters of t-RES are summarized in Tables 12A and 12B.

TABLES 12A and 12B
Plasma pharmacokinetic values following supplementation with RGC

| | | |
|---|---|---|
| Mean AUCₜ | Mean Cₘₐₓ | |
| (ng · hr/ml) | (ng/ml) | Median Tₘₐₓ |
| (% CV) | (% CV) | (hrs) [range] |

A. Total resveratrol

| | | | |
|---|---|---|---|
| RGC 150 mg (n = 15) | 10404 (29.9) | 1684 (33.1) | 4.00 [0.67-6.00] |
| RGC 50 mg (n = 15) | 2694 (52) | 458.4 (52.4) | 1.00 [0.33-8.00] |

B. Free resveratrol

| | | | |
|---|---|---|---|
| RGC 150 mg (n = 15) | 9.85 (74.3) | 6.89 (56.9) | 1 [0.33-4.00] |

Analyzing the first two time points of measurement, 0.33 and 0.67 hrs, reveals measurable quantities of RES in plasma of subjects received RGC (Table 13). Moreover, during the 0.33 hrs time point all subjects in the RGC groups (except one in the RGC 150 mg group) had measurable concentration of total RES (Tables 13 and 14).

TABLE 13
Subjects with detectable amounts of RES in plasma (total/free)
during the first two time points of measurement.

| Time point (hr) | |
|---|---|
| 0.33 | 0.67 |

| | Total | Free | Total | Free |
|---|---|---|---|---|
| RGC 150 mg | 14/15 | 11/15 | 15/15 | 13/15 |
| RGC 50 mg | 15/15 | | 15/15 | |

TABLE 14
Concentration (ng/mL) of total and free RES at the first
three time points of measurement after dosing.

| | Time point (hr) | | | | | |
|---|---|---|---|---|---|---|
| | 0.33 | | 0.67 | | 1 | |
| | Total | Free | Total | Free | Total | Free |
| RGC 150 mg | 248.9 ± 228 | 2.34 ± 3.5 | 906.6 ± 628 | 2.88 ± 2.5 | 1137 ± 640 | 1.21 ± 3.0 |

### CONCLUSIONS

RES originated in RGC is characterized by the addition of one hexose moiety. Although the exact type of hexose and its precise location have not been identified it is most likely that RGC RES is piceid—the most common type of RES occurs naturally in red grapes. The phenomenon of two peaks demonstrated both in the free and total forms of RES is unique and has not been observed in other types of RES. It is possible that the presence of a glycoside group in RGC RES rendered it more soluble, as can be observed in the solubility test in which RGC-RES was more water soluble that synesthetic and plant derived sources of RES This unique two concentration peaks pattern may also attributed to the unique composition of RGC that contains a whole matrix of red grapes polyphenols and high level of glycoside resveratrol and the synergism between them, This trait may have found expression in the high rate of presence of RES in high concentration as fast as 20 minutes after administration.

As clearly seen in the concentration/time curve (FIG. 6A), RGC-RES yielded two very distinct concentration peaks appearing at 1 and 5 hours.

Importantly, concentration time curves of synthetic or yeast fermentation sources of RES (Poulsen, M M., Vestergaard, P F., Clasen, B F., Radko, Y., et al., High-dose resveratrol supplementation in obese men: an investigator-initiated, randomized, placebo-controlled clinical trial of substrate metabolism, insulin sensitivity, and body composition. Diabetes 2013, 62, 1186-1195) as well as of plant derived RES (Amiot, M. J., Romier, B., Dao, T. M., Fanciullino, R., et al., Optimization of trans-Resveratrol bioavailability for human therapy. Biochimie 2013, 95, 1233-1238 show a distinct single concentration peak. This shows that one supplementation of RGC a day is enough for a prolonged effect, while in other products more than one supplementation or higher levels a day may be required for the same effect.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A large scale process for the in vitro production of a cell line callus culture of grape berry cells grown comprising:
growing grape cells in a flask;
inoculating the grape cells from the flask into a first bioreactor;
inoculating the grape cells from the first bioreactor into another bioreactor, wherein the another bioreactor is a last bioreactor or an intermediate bioreactor and wherein at least one of the first and the another bioreactor is disposable; and
harvesting the grape cells from the last bioreactor;
wherein the grape cells harvested from the last bioreactor are dried.

2. The large scale process of claim 1, wherein the size of each bioreactor used in the process is larger than the one in which the grape cells were previously grown.

3. The process of claim 1, wherein if the another bioreactor is an intermediate bioreactor, an additional step of inoculating the grape cells to another intermediate bioreactor or to the last bioreactor in performed.

4. The process of claim 1, wherein the first or another bioreactor is a 4-10 liter bioreactor.

5. The process of claim 1, wherein the first or another bioreactor is a 10-50 liter bioreactor.

6. The process of claim 1, wherein the first or another bioreactor is a 50-200 liter bioreactor.

7. The process of claim 1, wherein the first or another bioreactor is a 200-500 liter bioreactor.

8. The process of claim 1, wherein the first or another bioreactor is a 200-1000 liter bioreactor.

9. The process of claim 1, wherein the grape cells are grown in a Gamborg B5 medium.

10. The process of claim 9, wherein the Gamborg B5 medium is enriched with magnesium, phosphate or nitrate salts or a combination thereof.

11. The process of claim 9, wherein the Gamborg B5 medium is enriched with KNO₃, MgSO₄, MgNO₃ or NaH₂PO₄ or a combination thereof.

12. The process of claim 1, wherein the disposable bioreactor is made from one or more layers of polyethylene.

13. The process of claim 12, wherein the disposable bioreactor is made from an inner and outer layer of polyethylene and a middle nylon layer.

14. The process of claim 8, wherein the Gamborg B5 medium does not include plant hormones.

15. The process of claim 8, wherein the Gamborg B5 medium includes plant hormones.

16. The process of claim 9, wherein the Gamborg B5 medium is enriched with 2-4% sucrose.

17. A composition in a form of a powder comprising a cell line callus culture of grape berry cells grown in vitro in a large scale up process, whereby the cell line callus culture of grape berry cells is derived from one or more of grape-berry cross section, grape-berry skin, grape-berry flesh, grape seed, grape embryo of seeded or seedless cultivars or grape seed coat; wherein the cell line callus culture of grape berry cells includes resveratrol in an amount of at least 1000 mg/kg powder.

18. The composition of claim 17, characterized by two peaks of concentration of resveratrol in the plasma following a single administration of the composition.

19. A composition comprising a complex of pholyphenols including resveratrol, wherein the ratio of the resveratrol to the pholyphenols is higher than 1:20.
